(19) United States
(12) Patent Application Publication
(10) Pub. No.: US 2011/0257671 A1
(43) Pub. Date: Oct. 20, 2011

(54) CONTACT DETECTION TUBES, METHODS AND SYSTEMS FOR MEDICAL PROCEDURES

(75) Inventors: Karen Irene Trovato, Putnam Valley, NY (US); Wouter Anthon Soer, Nijmegen (NL)

(73) Assignee: KONINKLIJKE PHILIPS ELECTRONICS N.V., EINDHOVEN (NL)

(21) Appl. No.: 13/141,350

(22) PCT Filed: Nov. 10, 2009
(86) PCT No.: PCT/IB2009/054990

§ 371 Date: Jun. 22, 2011

(30) Foreign Application Priority Data
Dec. 22, 2008 (US) 61139696

(51) Int. Cl. A61B 17/34

(52) U.S. Cl. 606/185

(57) ABSTRACT

A contact detection method (70) involves a navigation of a contact detection tube (20′) within an open space of an anatomical region (50) of a body. The contact detection tube includes a tubular wall (21) having an interior surface (23) defining a working channel (24), and an electrode (30) integrated in the tubular wall (21). The electrode (30) electrically connects the contact detection tube (20′) to an electrically conductive object (41, 52) (e.g., biological tissue or a medical instrument/tool) in physical contact with an exterior surface (22) of the tubular wall (21) and electrically isolates the working channel (24) from any electrical connection of the tube (20′) to the object (41, 52). The method (70) further involves a determination of a contact status of the contact detection tube (20′) between an open state (i.e., no physical contact) and a closed state (i.e., physical contact).

The present invention generally relates to minimally invasive surgical procedures and specifically relates to contact detection tubes for minimizing any damage to internal body tissue during a surgical procedure.

Active Cannulas as known in the art are a set of telescoping, pre-shaped Nitinol tubes intended to be extended with each tube having a particular pre-designed curvature. Nitinol's ‘perfect memory’ enables the tube to straighten or conform inside the larger tube surrounding it until it is extended. The original intent of active cannulas was to rely on the interaction of the physical strength between two tubes to create motion, such as, for example, when a curved tube rotated within another curved tube. A more recent use for active cannulas described in International Publication no. WO 2008/032230 A1 to Trovato et al. published Mar. 20, 2008, and entitled “Active Cannula Configuration for Minimally Invasive Surgery” is to introduce the tubes sequentially. The largest tube is first extended, followed by each successively smaller tube. A specialized non-holonomic path planning computation determines the proper shape and length of each tube so that the cannulas can be sequentially deployed through complex anatomy, yet reach a defined destination at a particular orientation.

The active cannula must be configured in a specific way to reach a target, while avoiding anatomical ‘obstacles’. A technique that can design the configuration of a device having many controlled degrees of freedom is described in International Publication WO 2007/042986 A2 by Trovato et al. published Apr. 17, 2007, and entitled “3D Tool Path Planning, Simulation and Control System”. This technique can be used to configure a nested cannula, bronchoscope and steerable needle.

Although the tube lengths and curvatures can be specified precisely, miniaturization of many tubes can cause handling problems. Smaller sizes reach 0.007 inches (0.2 mm) which challenges the configuration of the devices as well as deployment. Manual deployment of tiny Active Cannula tube sets can be performed to reach a fixed location, such as, for example, as described in PCT/IB ______filed ______, published ______ as WO ______, claiming priority from U.S. provisional application No. 61/038,225, filed Mar. 20, 2008, the entirety of which is incorporated herein by reference. The blocks are specifically set to a given point each tube, and at a precise orientation, so that when they slide together, they reach the desired location. The blocks and supporting track provide easier handling of blocks and the ability to reach a final target.

An automated electromechanical device for precision deployment of a series of Active Cannulas is described in prior, co-pending U.S. provisional application No. 61/098,233, filed Sep. 16, 2008, which was filed ______ as PCT/IB ______ and published ______ as WO ______, the entirety of which is incorporated herein by reference. This automated system enables a single set of cannulas to be redeployed in numerous different configurations, with automated, repeatable precision. Such a system can be used in particular for laparoscopic procedures.

Many tools can also be delivered endoscopically such as biopsy needles, cautery and ligation devices, scissors, snares, baskets, etc. As they are advanced into the patient, it can be difficult to know when these tools first come in contact with tissue rather than relying on tactile or visual validation, which may be imprecise.

Safety is of paramount importance when using a device within the human body. Pre-planning and simulation can work well in a highly constrained environment such as in manufacturing or even for radiation oncology where the patient is immobilized. In surgery however, patient motion, breathing for example, is a potential problem. The use of multiple tools might also cause incidental motion if they collide or obstruct the surgeon's view.

Each new technology must take care ‘to do no harm’. Once the machinery has been used and modeled extensively, trust, as a result of experience and understanding, will move the technology to the complementary term ‘tool’. Until that time however, safeguard mechanisms must be in place. Devices operated at a distance such as in endoscopy, have further complications such as counterintuitive and sometimes non-linear control, 2D rather than 3D imaging, little tactile feedback etc.

The safety systems known in the art are unworkable for active cannula and the like employing invasive tubes. It is desirable to have sensory feedback for devices such as the nested cannula.

For example, surgical robots such as Intuitive's daVinci robot, rely on the judgment and dexterity of the surgeon for safe operation. Hand-eye coordination is important, and is improved by 10× magnification of the surgical field. The system relies on the assumption that motions performed by the surgeon's hands are perfectly replicated electronically, by the robot.

Other mechanisms providing safety for current robots include rigorous simulation to assess safe motions. Watchdog circuits can be used to cut motion when a particular limit switch is activated, or a specific torque is exceeded. This is typically performed in a custom manner to detect when the machine has exceeded a pre-defined configuration (such as rotation beyond a particular angle), indicating a dangerous pose. Watchdogs typically require limit switches such as laser or mechanical that are carefully placed on joints or on edges of the robot work envelope. For laparoscopic procedures, laser lights are impractical because they cannot be set and maintained. Limit switches are ‘per joint’. Although small, they are much larger than a Nested Cannula. Finally, limit switches do not necessarily correspond to collisions that might occur at the tool tip, nor through the body of the device, most especially if the device is not a rigid. Another safety device is a collision sensor, such as the spring-loaded Uni-Coupler. This device is mounted on a robot, creating a flexible (compliant) joint that triggers a ‘stop’ signal to the system if the tool tip collides unexpectedly, with sufficient force.

Virtual Fixtures are a class of control mechanisms, useful for tele-robotics that allows permitted working areas to be defined where control directives to move into an illegal area are rejected. A forbidden-region virtual fixture (FRVF) is known as a computer-generated constraint that provides position or force limits to a robot manipulator or operator, in order to prevent motion into forbidden regions of the workspace. One could imagine a Virtual Fixture in terms of 4 planes that virtually box-in the permitted motions as though there were physical walls guiding the motion.

Electromagnetic tracking is a known method for determining the position and orientation of an object using tiny electromagnetic coils to detect EM field strength. Tracking components are available from companies such as NDI, which make the Aurora system. An example tracking product is Traxtal's PercuNav system.

There are many devices to detect continuity of a circuit. Common, typically inexpensive devices include a multi-meter or continuity tester. A multi-meter can be used to measure resistance in ohms (Ω). When the resistance is infinity, or very high, the circuit is ‘open’ and cannot conduct electricity, otherwise it is ‘closed’. A continuity tester is in essence, a battery, often a 9 volt or AA (1.5 volt), and a light bulb or buzzer. Each device has two wires that connect opposite sides of the circuit to be tested.

Materials used for insulating electrical circuits are well known, and include but are not limited to: polyethylene, PVC (polyvinyl chloride), polycarbonate, rubber-like polymers, Teflon, silicone, and many others.

There are also switches that detect a change in radio wave reception, which changes when the length of the ‘antenna’ varies, such as when it comes into contact with another conductor, such as the body.

Touch sensitive lamps detect changes in capacitance to determine that a lamp base for example, has been touched. Based on the change in capacitance, the control circuitry signals a change in light output.

The present invention improves safety by providing a safety mechanism integrated within a minimally invasive tube that does not interfere or comprise the operation of the minimally invasive tube.

One form of the present invention is a contact detection method involving a navigating of a contact detection tube within an open space of an anatomical region of a body, the contact detection tube including a tubular wall having an interior surface defining a working channel, and an electrode integrated in the tubular wall. In operation, the electrode electrically connects the contact detection tube to an electrically conductive object within the anatomical region in physical contact with an exterior surface of the tubular wall, and the electrode electrically isolates the working channel from any electrical connection of the contact detection tube to the electrically conductive object. The method further involves a determination of an electrical contact status of the contact detection tube between an open state and a closed state. The open state is representative of a sensing an open circuit between the contact detection tube and the electrically conductive object, and the closed state is representative of a sensing of a closed circuit between the contact detection tube and the electrically conductive object.

For purposes of the present invention, the term “electrically conductive object” is broadly defined herein as any object within an anatomical region of a body that is materially capable of facilitating a measureable current flow, direct or alternating, through the object. Examples of an electrically conductive object include, but are not limited to, biological tissue (e.g., skin and internal organs) and medical instruments, tools and devices of any kind.

A second form of the present invention is a nested cannula set employing a plurality of telescoping tubes configured and dimensioned to reach a target location relative to an anatomical region of a body wherein the nested cannula set incorporates one or more of the aforementioned contact detection tube.

A third form of the present invention is a contact detection system employing the aforementioned contact detection tube and a contact sensing device. In operation, the contact sensing device senses the contact status of the contact detection tube between the open state and the closed state.

The foregoing forms and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

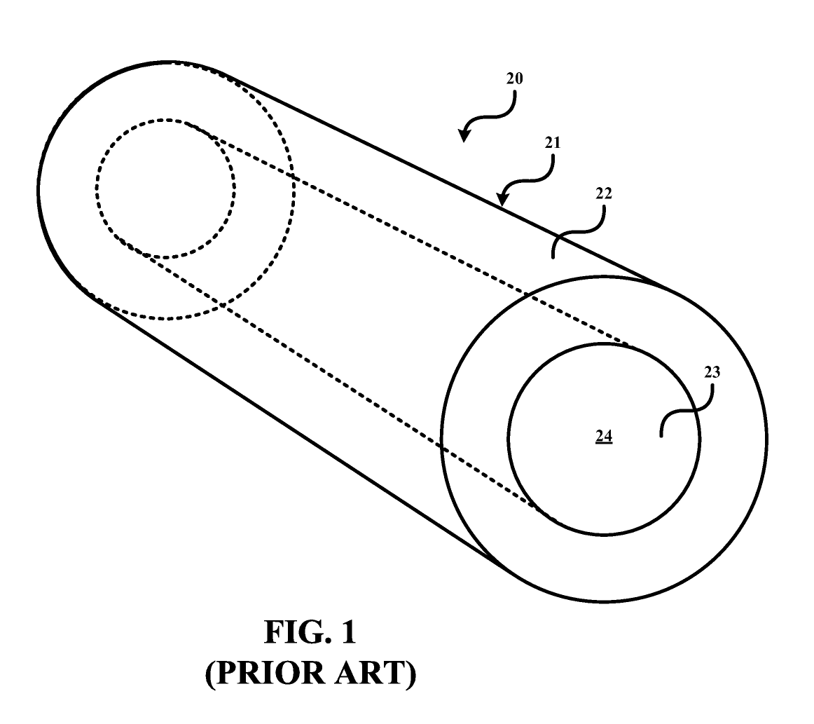

FIG. 1 illustrates a minimally invasive tube employed in medical instruments as known in the art.

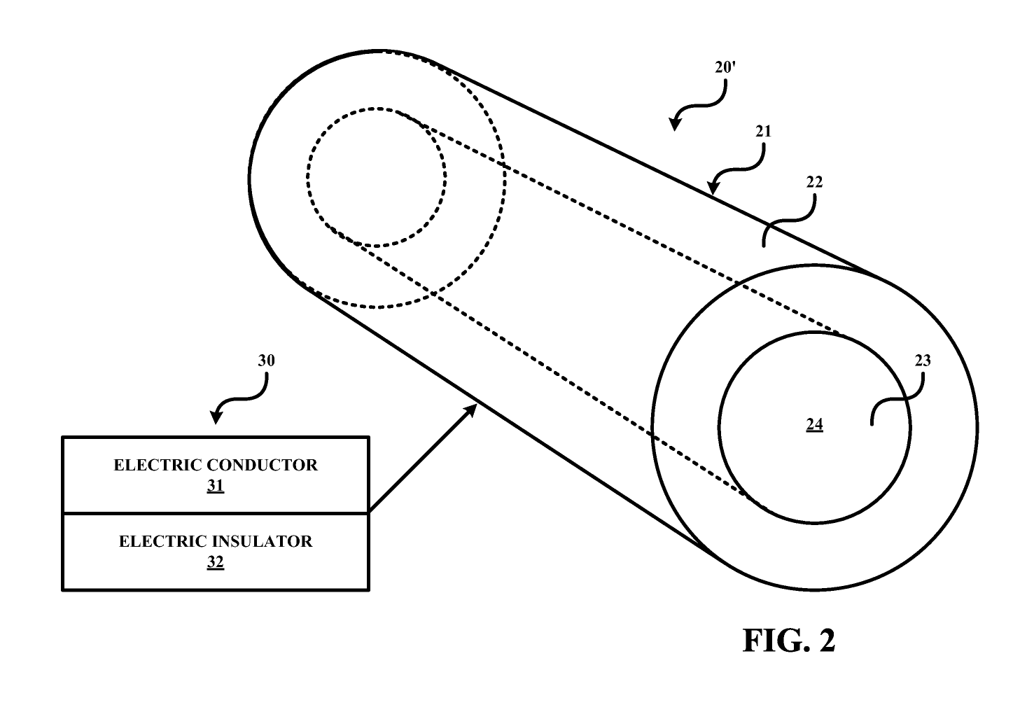

FIG. 2 illustrates an exemplary minimally invasive contact detection tube for employment in medical instruments in accordance with the present invention.

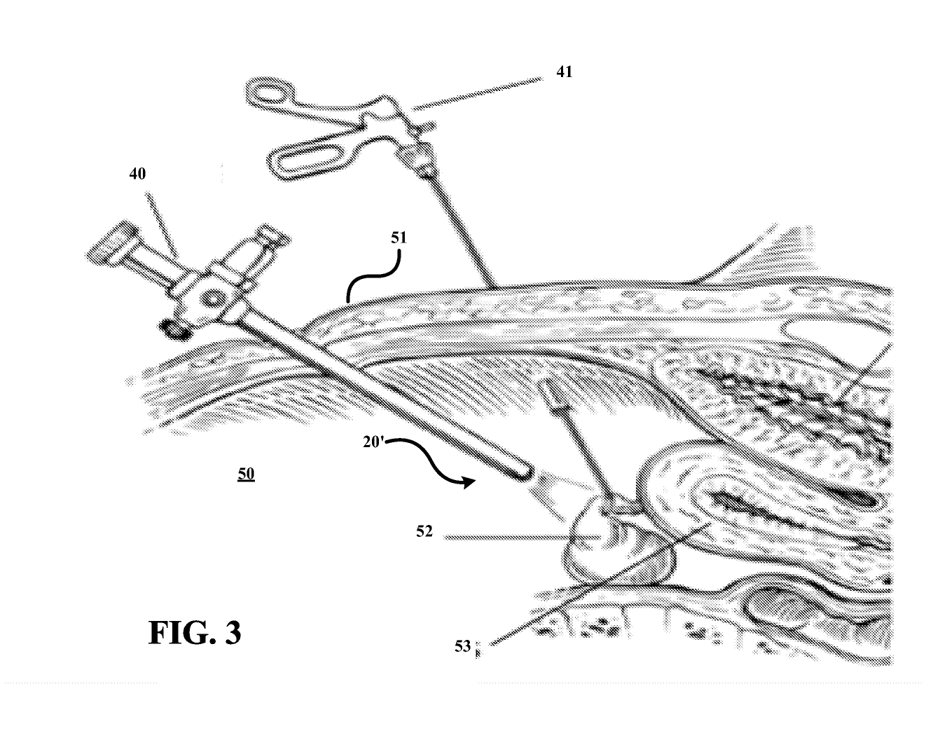

FIG. 3 illustrates a laparoscopic procedure involving a minimally invasive contact detection tube in accordance with the present invention.

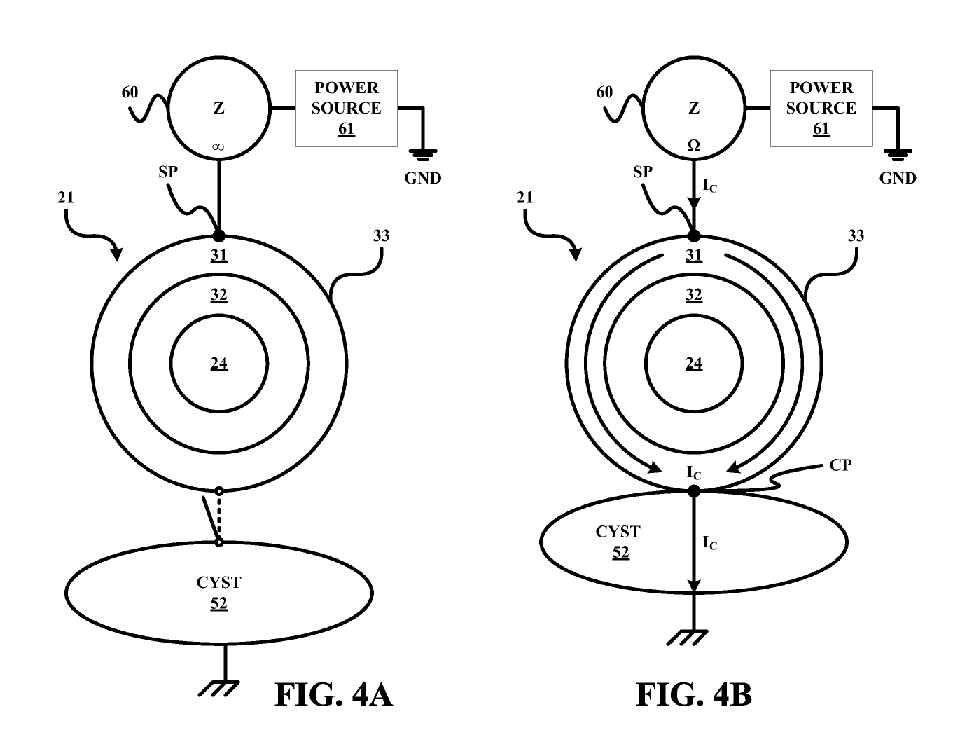

FIGS. 4A and 4B respectively illustrate an open state and a closed state of the minimally invasive contact detection tube illustrated in FIG. 3 as related to a cyst illustrated in FIG. 3.

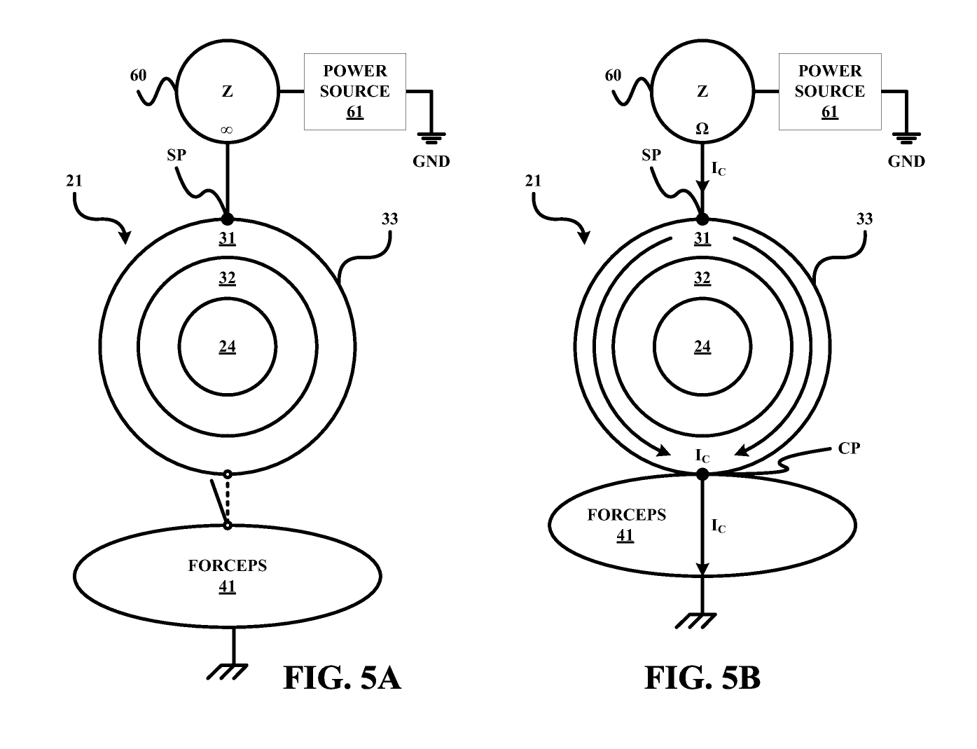

FIGS. 5A and 5B respectively illustrate a open state and a closed state of the minimally invasive tube illustrated in FIG. 3 as related to a forceps illustrated in FIG. 3.

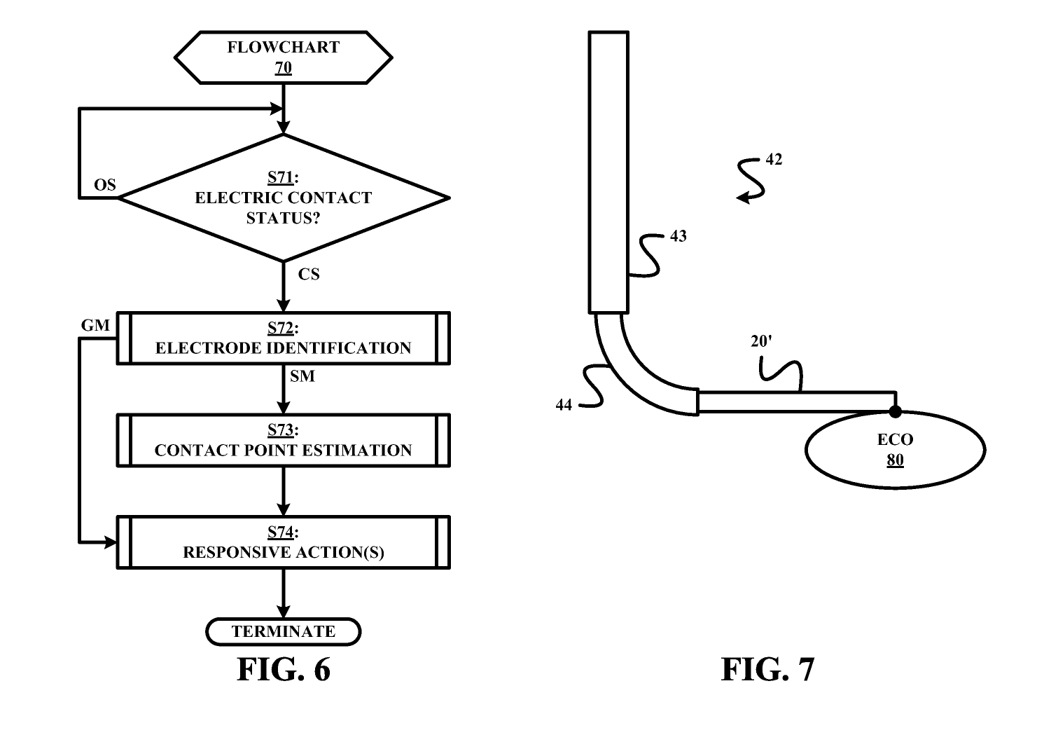

FIG. 6 illustrates a flowchart representative of an exemplary embodiment of a contact detection method in accordance with the present invention.

FIG. 7 illustrates an exemplary physical contact between a minimally invasive contact detection tube of the present invention and a bronchial tree.

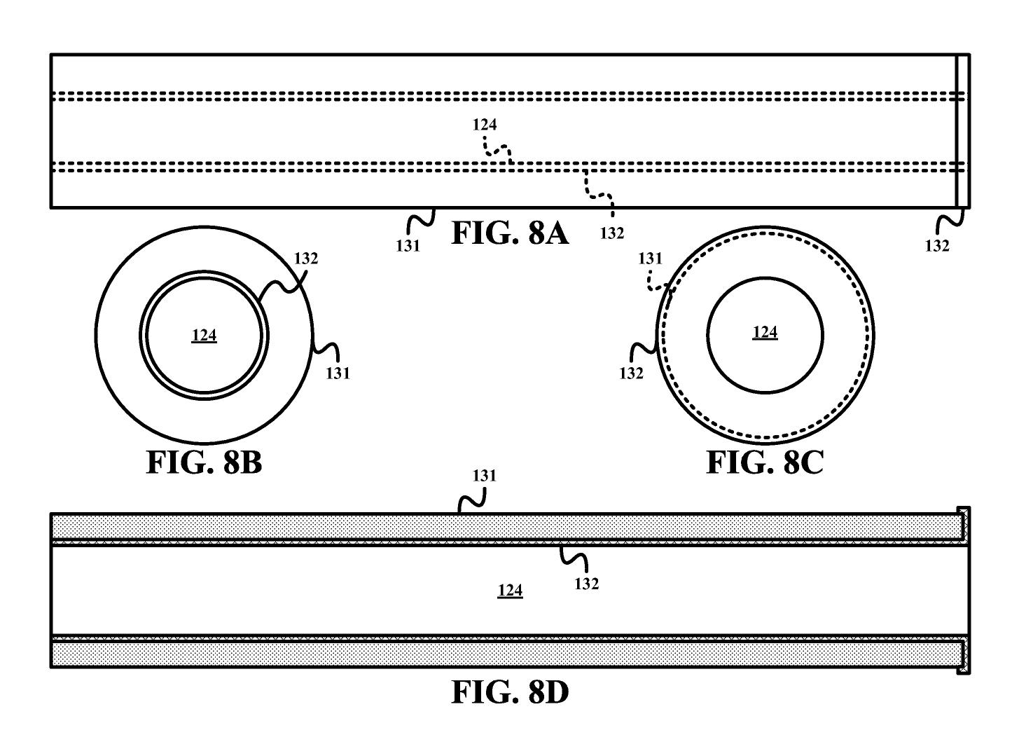

FIGS. 8A-8D respectively illustrate a side view, a proximal view, a distal view and a cross-sectional view of a first exemplarily embodiment of the minimally invasive contact detection tube illustrated in FIG. 2 in accordance with the present invention.

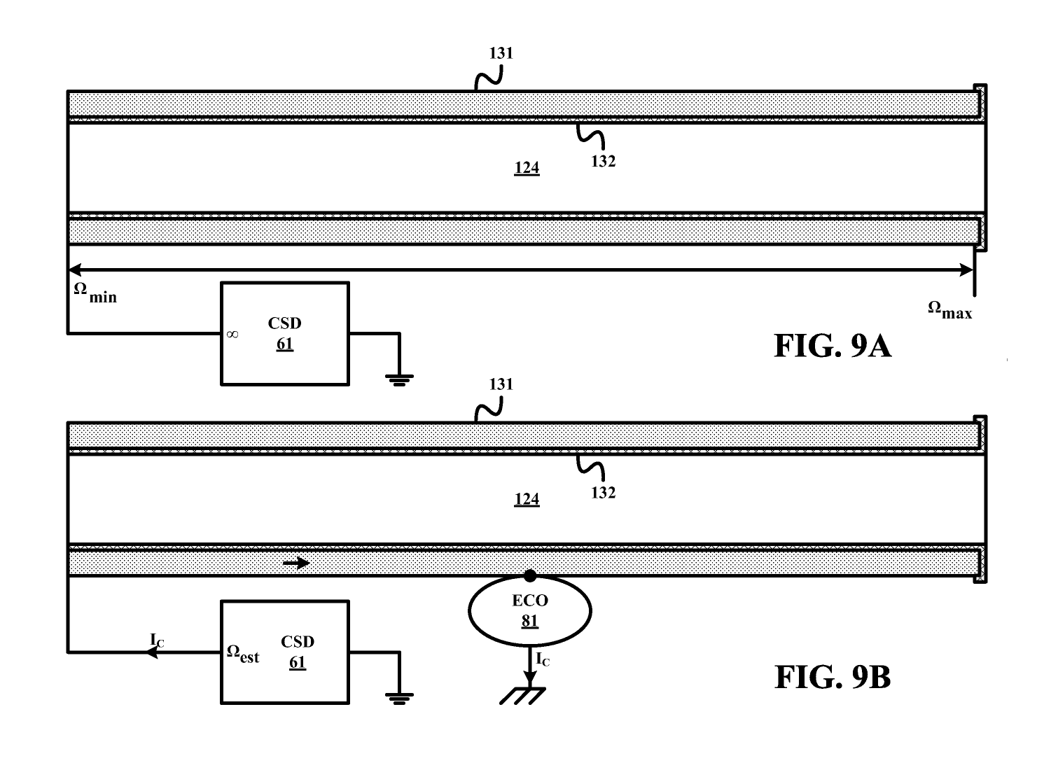

FIGS. 9A and 9B illustrate operational characteristics of the minimally invasive contact detection tube illustrated in FIGS. 8A-8D.

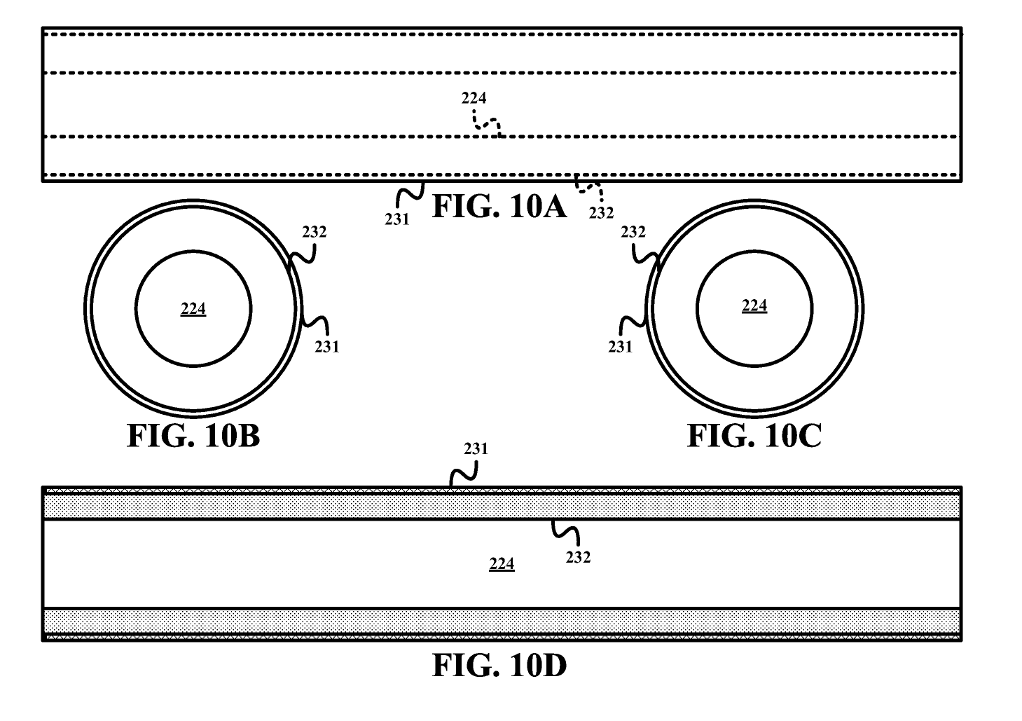

FIGS. 10A-10D respectively illustrate a side view, a proximal view, a distal view and a cross-sectional view of a second exemplarily embodiment of the minimally invasive contact detection tube illustrated in FIG. 2 in accordance with the present invention.

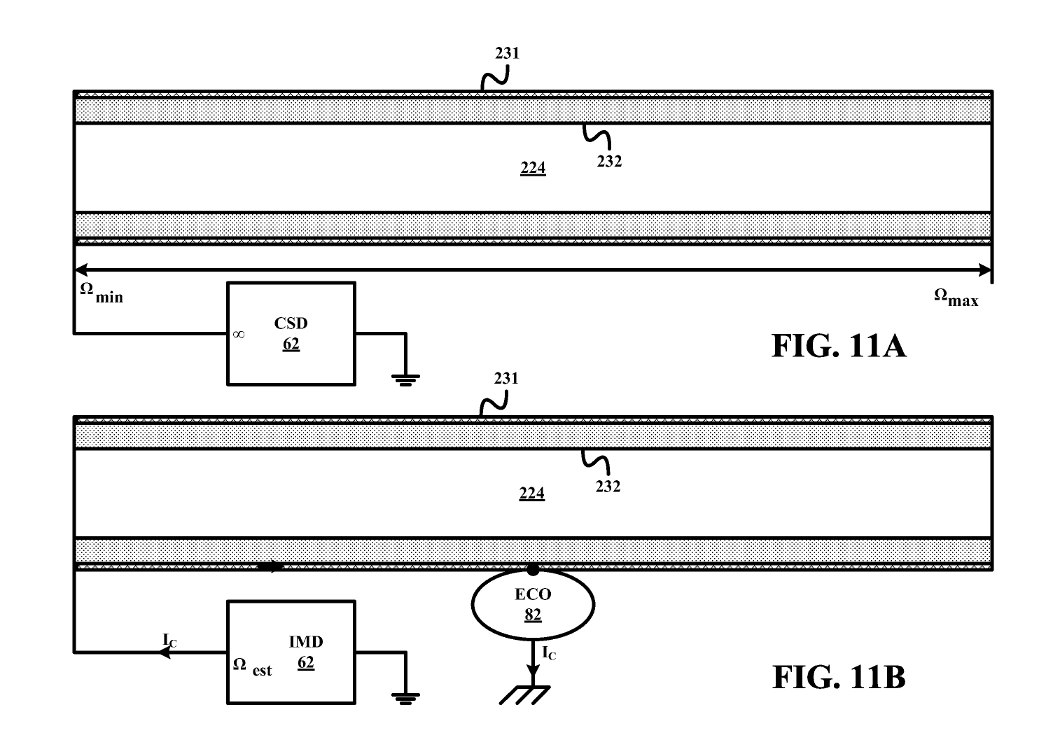

FIGS. 11A and 11B illustrate operational characteristics of the minimally invasive contact detection tube illustrated in FIGS. 10A-10D.

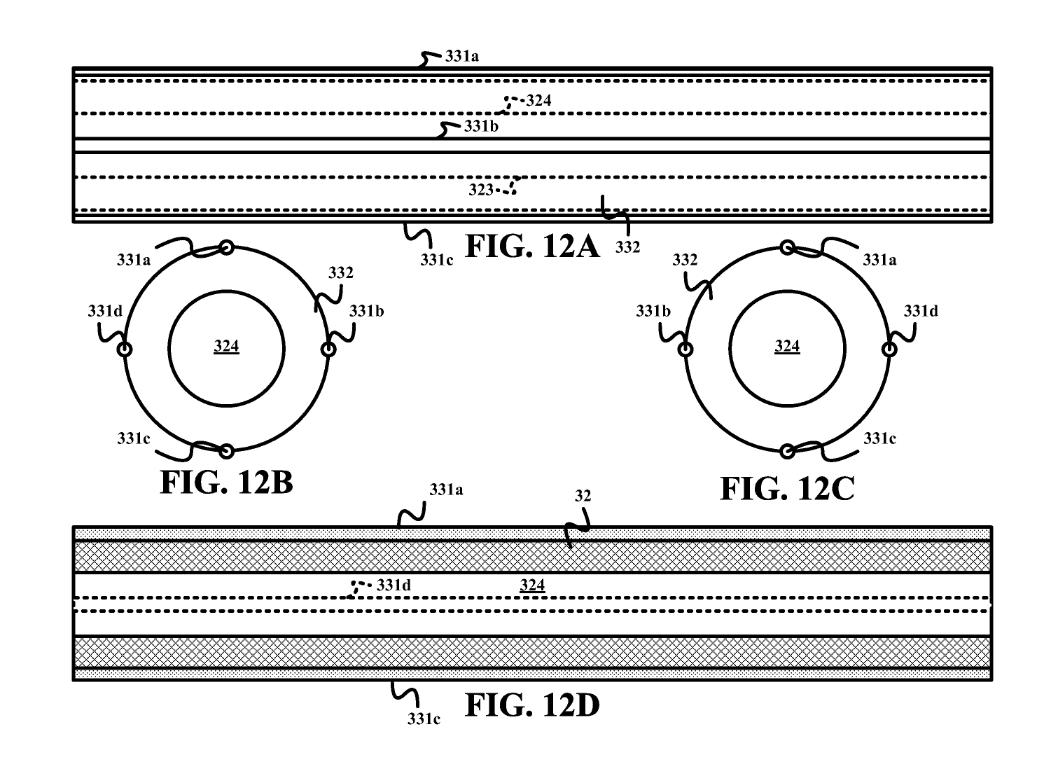

FIGS. 12A-12D respectively illustrate a side view, a proximal view, a distal view and a cross-sectional view of a third exemplarily embodiment of the minimally invasive contact detection tube illustrated in FIG. 2 in accordance with the present invention.

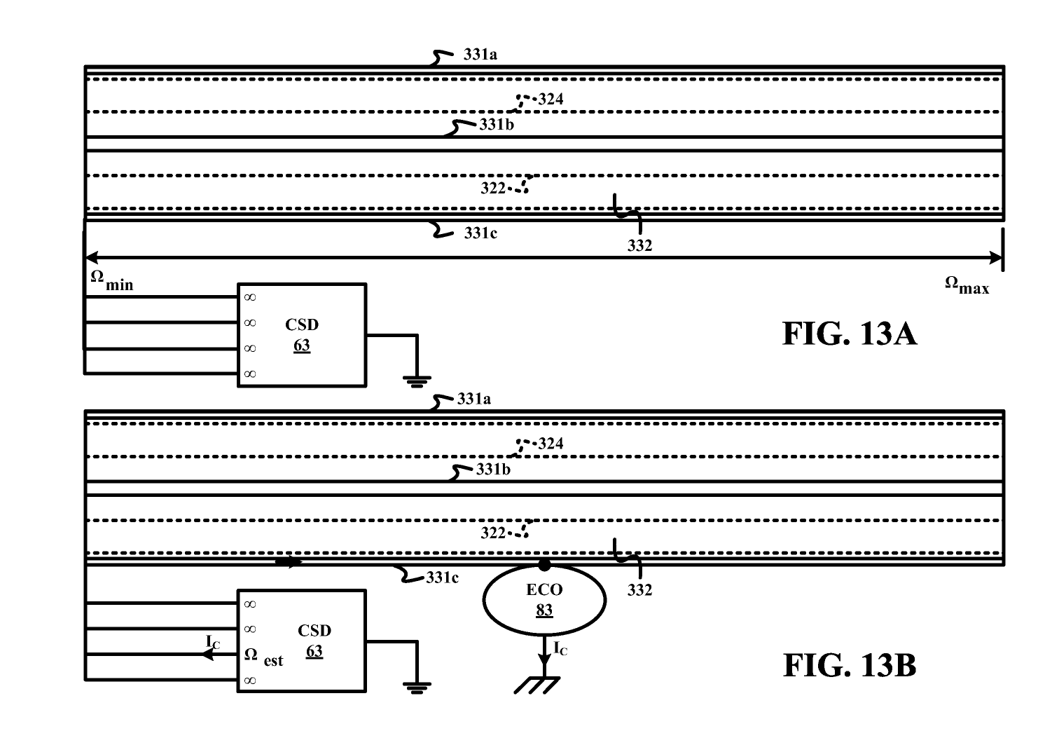

FIGS. 13A and 13B illustrate operational characteristics of the minimally invasive contact detection tube illustrated in FIGS. 12A-12D.

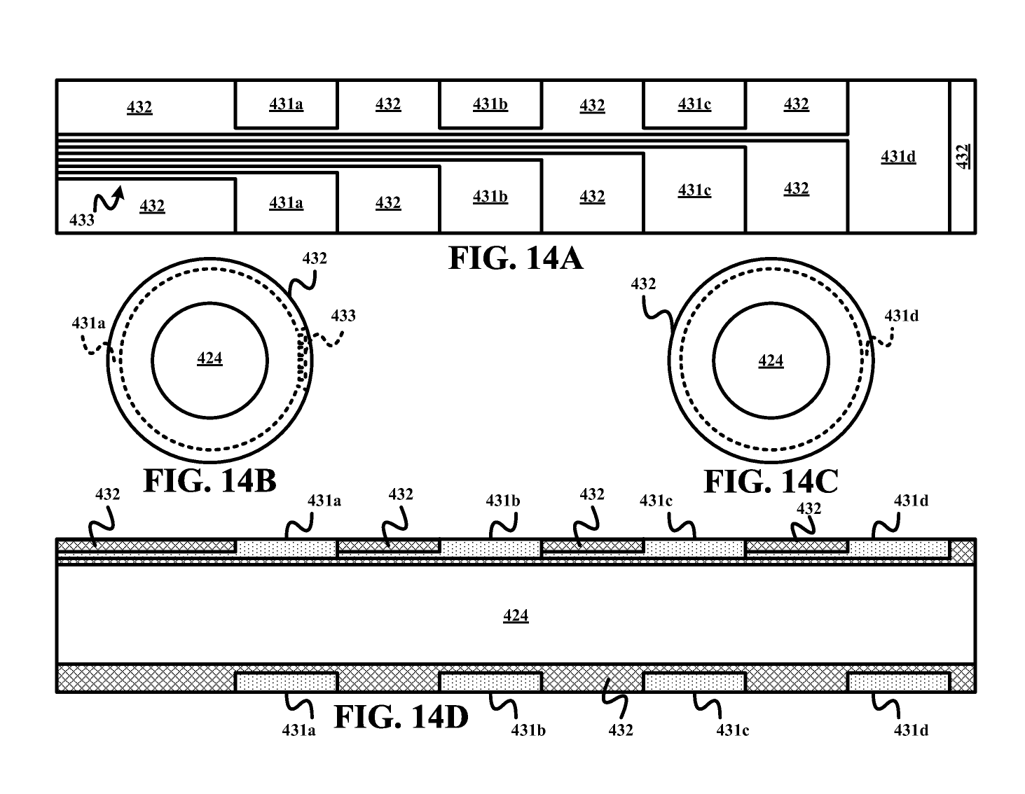

FIGS. 14A-14D respectively illustrate a side view, a proximal view, a distal view and a cross-sectional view of a fourth exemplarily embodiment of the minimally invasive contact detection tube illustrated in FIG. 2 in accordance with the present invention.

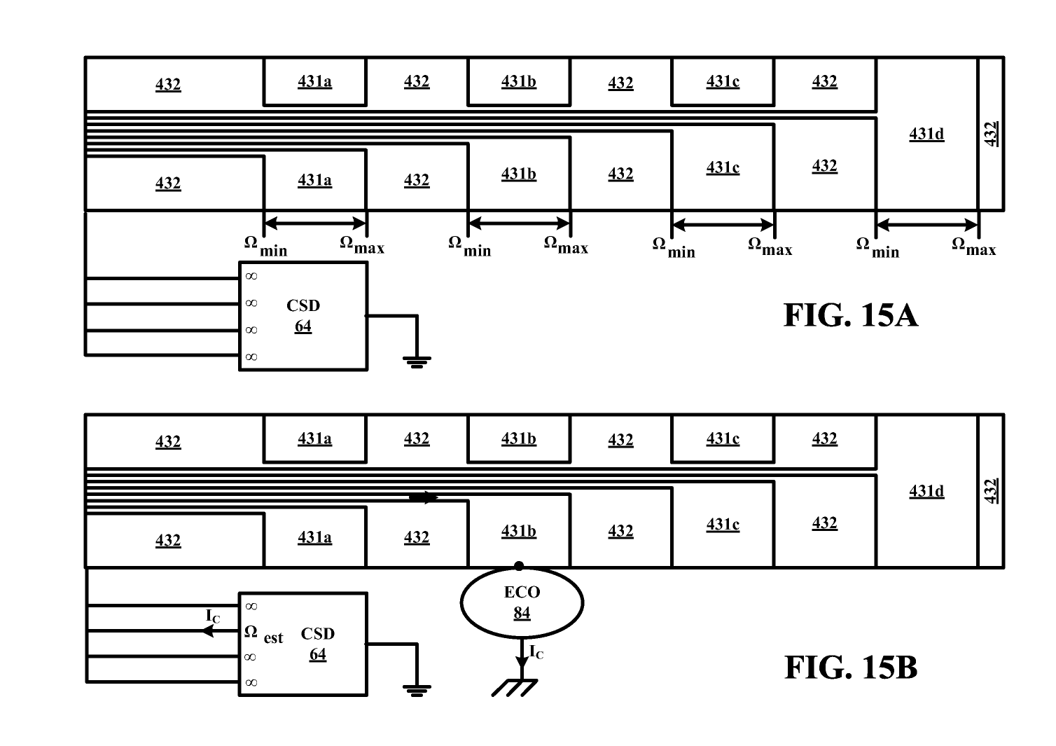

FIGS. 15A and 15B illustrate operational characteristics of the minimally invasive contact detection tube illustrated in FIGS. 14A-14D.

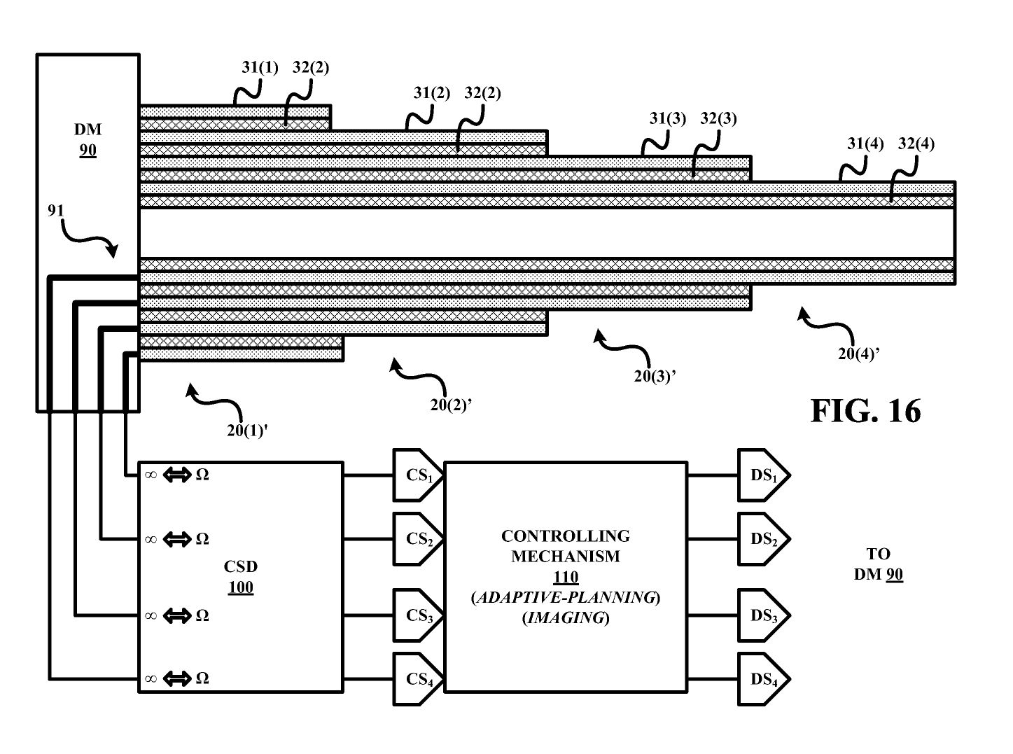

FIG. 16 illustrates an exemplary embodiment of a contact detection system in accordance with the present invention.

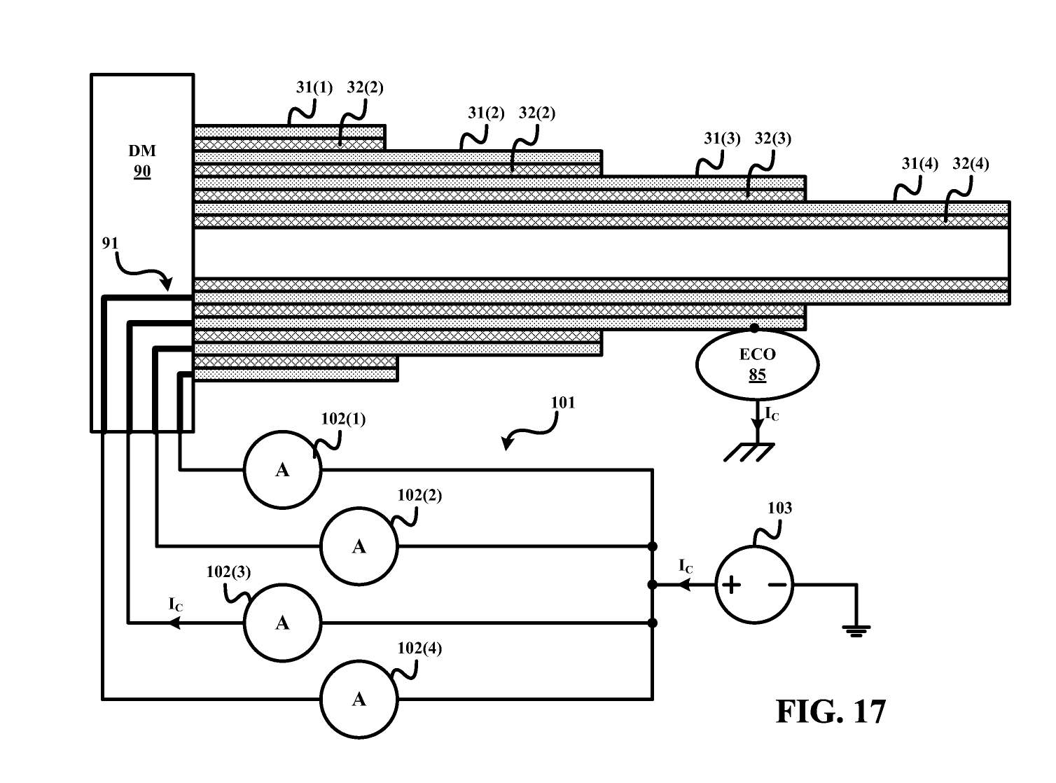

FIG. 17 illustrates a first exemplary embodiment of a contact sensing device illustrated in FIG. 16 in accordance with the present invention.

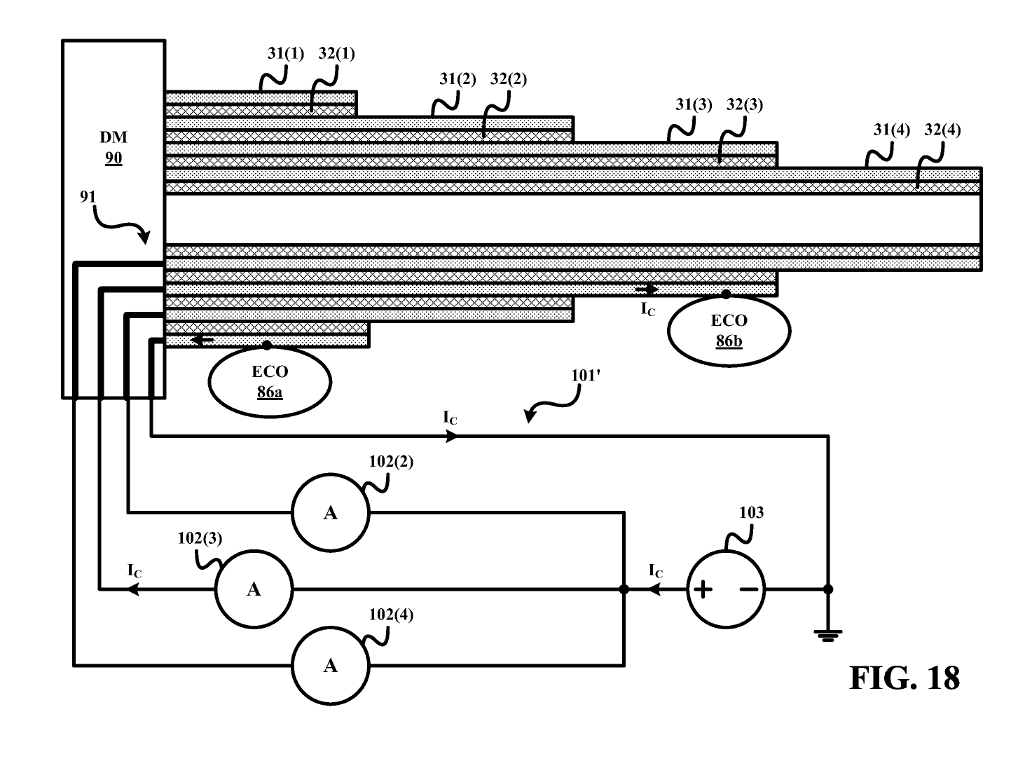

FIG. 18 illustrates a second exemplary embodiment of a contact sensing device illustrated in FIG. 16 in accordance with the present invention.

FIG. 1 illustrates a minimally invasive tube 20 for a medical instrument (e.g., a catheter, an endoscope, an active cannula, a nested cannula, etc.) that facilitates an introduction of a medical tool (e.g., a surgical tool or an endoscopic tool) within a body, human or animal. To this end, tube 20 has a tubular wall configured and dimensioned to be manually or mechanically navigated within an anatomical region of a body. Tubular wall 21 has an interior surface 23 defining a working channel 24 for insertion of the medical tool or another tube 20 (smaller, not shown) to facilitate the medical tool reaching a target location within the body. In order to aid the introduction of one or more tubes 20 within the body while preventing any inadvertent tissue damage, the present invention is premised on a detection of internal biological tissue physically contacting an exterior surface 22 of tubular wall 21 by determining an electrical contact status of tubular wall 21. Generally, as shown in FIG. 2, this is achieved in accordance with the present invention by integrating one or more electrodes 30 in tubular wall 21 to yield a contact detection tube 20′. Specifically, each electrode 30 has an electric conductor 31 as known in the art and an electric insulator 32 as known in the art that are integrated into tubular wall 21 in any manner that makes a portion or an entirety of exterior surface 22 of tubular wall 21 electrically conductive and completely electrically insulates working channel 24 from the electrical conductivity of tubular wall 22.

For example, FIG. 3 illustrates a laparoscopic procedure involving a laparoscope 40 and a laparoscopic grasping forceps 41 inserted through incisions of a skin tissue 51 leading into an abdominal cavity 50. As shown, laparoscope 40 utilizes a fiber-optic light to illuminate a cyst 52 attached to a uterus 53 whereby forceps 41 may be used to remove cyst 52 from abdominal cavity 50. In this case, laparoscope 40 has been equipped with a contact detection tube 20′ at its distal end to facilitate a determination when laparoscope 40 is in physical contact with cyst 52 or forceps 41.

More particularly, FIGS. 4A and 4B show contact detection tube 20′ including an outer tubular electric conductor 31 and an inner tubular electric insulator 32 whereby the inner surface of the electric insulator 32 defines working channel 24. A very small amount of power, direct or alternating, is applied to the outer tubular electric conductor 31, and as in all simple electric circuits, there must be a conducting return path to form (close) the circuit. This is often called ‘ground’, denoting a ubiquitous connected conductor. In our case ‘ground’ is the patient's body since human tissue is generally conductive, but it may include conducting objects in contact with the patient, such as metal tools or a table. Thus, when contact is made between the outer tubular electric conductor 31 and cyst 52, the circuit is closed, resulting in a measurable electrical current flow. As related to cyst 52, the electrical contact status of contact detection tube 20′ is either an open state as shown in FIG. 4A, or a closed state as shown in FIG. 4B.

Referring to FIG. 4A, the open state is representative of an open circuit detected by a contact sensing device 60, which is conductively coupled to a power source 61 (direct or alternating) and outer tubular electric conductor 31. Specifically, the absence of any physical contact between outer tubular electric conductor 31 and cyst 52, the circuit is ‘open’, and in this case, device 60 senses an infinite impedance ∞ at a sensing point SP of conductor 31. This measurement indicates that contact detection tube 20′ is not directly or indirectly in contact with cyst 52.

Conversely, referring to FIG. 4B, the closed state is representative of a closed circuit detected by contact sensing device 60. Specifically, physical contact between outer tubular electric conductor 31 and cyst 52 closes the circuit and in this case, contact sensing device 60 senses measurable resistance Ω below ∞ at a sensing point SP of conductor 31. This measurement indicates that contact detection tube 20′ is directly or indirectly in contact with cyst 52.

Additionally, working channel 24 is electrically isolated from a current I_{C} to prevent any electrical interference with any biological tissue and/or medical tool inserted within working channel 24, and/or with any additional tube 20 or 20′ nested within working channel 24.

The measurable impedance Ω is a total impedance Z_{T} of the circuit that is determined by the impedance Z_{C} of conductor 31 and the object impedance Z_{O} of cyst 52.

In a general measuring mode of tube 20′, the conductor impedance Z_{C} relative to the object impedance Zₒ does not facilitate an estimation of the conductor impedance Z_{C} between sensing point SP and connection point CP. For example, the conductor impedance Z_{C} being equal to or less than the object impedance Z_{O} does not facilitate a measurement calculation of conductor impedance Z_{C} between sensing point SP and connection point CP. Also by example, the conductor impedance Z_{C} being greater than the object impedance Z_{O} to a degree where Z_{T}≠ Z_{C} does not facilitate a measurement calculation of conductor impedance Z_{C} between sensing point SP and connection point CP. The general measuring mode is therefore useful to detect the physical contact between conductor 31 and cyst 52 excluding an estimation of the location of contact point CP along an exterior surface 33 of conductor 31.

In a specific measuring mode of tube 20′, the conductor impedance Z_{C} relative to the object impedance Z_{O} facilitates a measurement calculation of the conductor impedance Z_{C} between sensing point SP and connection point CP in view of the conductor impedance Z_{C} being greater than the object impedance Z_{O} to a degree where Z_{T}≈Z_{C}. The specific measuring mode is therefore useful to detect the physical contact between conductor 31 and cyst 52 including an estimation to the location of contact point CP along exterior surface 33 of conductor 31 based on the conductor impedance Z_{C} of conductor 31 being function of a physical geometry and a material resistance and/or reactance of conductor 31 as known in the art.

As related to forceps 41, the electrical contact status of contact detection tube 20′ is either an open state as shown in FIG. 5A or a closed state as shown in FIG. 5B. In practice, contact sensing device 60 may not be structurally configured to differentiate between physical contact of the contact detection tube 20′ and cyst 52, and physical contact of the contact detection tube 20′ and forceps 41. However, if such differentiation is essential to the application of contact detection tube 20′, then various intra-operative imaging techniques may be implemented to facilitate whether contact detection tube 20′ is in physical contact with cyst 52 and/or forceps 41 as will be appreciated by those having ordinary skill in the art.

To further facilitate an understanding of the measuring modes as related to tube 20′, FIG. 6 illustrates a flowchart 70 representative of a contact detection method of the present invention as would be implemented on behalf of contact detection tube 20′ (FIG. 1). Specifically, a stage S71 of flowchart 70 encompasses a determination of the electrical contact status of contact detection tube 20′ between a open state (“OS”) as exemplarily shown in FIGS. 4A and 5A, and a closed state (“CS”) as exemplarily shown in FIGS. 4B and 5B. Flowchart 70 proceeds to a stage S72 of upon a determination that contact detection tube 20′ is in the closed state (“CS”).

Stage S72 encompasses an identification of the exact electrode or electrodes 30 of contact detection tube 20′ being physically contacted by an electrically conductive object (e.g., cyst 52 or forceps 41 shown in FIG. 3). The electrode identification is important for the execution of appropriate responsive action(s) during a stage S74 of flowchart 70. Particularly, it may be important to distinguish contact detection tube 20′ having a single integrated electrode 30 in physical contact with an electrically conductive object versus contact detection tube 20′ having multiple integrated electrodes 30 that may simultaneously or sequentially be in physical contact with the electrically conductive object.

In a general measuring mode (“GM”) of stage S72, the electrode(s) 30 of contact detection tube 20′ in physical contact with an electrically conductive object are identified and stage S74 is thereafter executed to implement any necessary responsive action(s) to prevent any inadvertent tissue damage by contact detection tube 20′, or if tissue damage is of no concern, to continue with the procedure in view of the physical contact. Stage S72 computes the estimated location of the contacting electrode(s) 30 relative to the entire tubular wall 21 and therefore will be able to provide an approximation of the location of each contact point CP between contact detection tube 20′ and the electrically conductive object.

For example, FIG. 7 shows a nested cannula set 42 having tubes 43 and 44 and a contact detection tube 20′ whereby contact detection tube 20′ has come into physical contact with an electrically conductive object 80 (e.g., body tissue) as contact detection tube 20′ was being extended from tube 44. When electric contact status becomes CS, indicating that contact detection tube 20′ has touched electrically conductive object 80, the controlling method may generate a signal. This signal may generate audible and/or visual feedback indicating physical contact between contact detection tube 20′ and electrically conductive object 80. Alternatively or concurrently, the signal may initiate a response, such as, for example, directing the tube controller (or user) to retract contact detection tube 20′ away from the physical contact.

In another example, if intra-operative imaging is being used to navigate contact detection tube 20′ based on a pre-operative plan and physical contact between contact detection tube 20′ and object 80 is detected, then image data may be acquired to approximate the location of the physical contact between contact detection tube 20′ and object 80 along the exterior surface of contact detection tube 20′. The location may be used for visual feedback to the user or as drive signals to selectively retract, advance and/or rotate contact detection tube 20′ in accordance with the pre-operative plan. The execution of the appropriate response is straightforward in the case of contact detection tube 20′ having a single integrated electrode 30, but may be more intensive in the case of contact detection tube 20′ having multiple integrated electrodes 30 as would be appreciated by those having ordinary skill in the art.

In a specific measuring mode (“SM”) of stage S72, prior to proceeding to stage S74, a stage 73 of flowchart 70 encompasses the computation of the location based on information from the contact sensing device. For example, referring to FIG. 7, while controlling nested cannula 42, contact detection tube 20′ is exposed only in certain areas along the path. The responsive action at S74 is determined by the application and the estimated contact position. For example, contact with contact detection tube 20′ could indicate any surface location along contact detection tube 20′ extended beyond tube 44. If the precise extension of each tube is known, then the surface area size and location of contact detection tube 20′ can be calculated. Naturally, the farther contact detection tube 20′ is extended, the more potential contact locations exist.

As a responsive action, the system may sequentially retract all of the tubes from smallest 20′ to intermediate 44 to largest 43, and then sequentially re-extend the tubes from largest 43 to intermediate 44 to smallest 20′ with at least one having a different length to reach in another direction. This can be performed most easily if the system is deployed with automatic control, but can be achieved manually as well.

In an alternative technique to maintain rather than avoid contact, contact detection tube 20′ may be extended until it touches object 80 as indicated by a closed state. If the electrical contact status thereafter become an open state indicating that contact is lost, then contact detection tube 20′ may be extend slowly until contact is restored. In the simplest case, a tube that indicates contact has a limited region for that possible contact.

For contact detection tube 20′, an estimated location can be computed for the contact point along the exterior surface 33 of a conductor 31 physically contacting the electrically conductive object. The controlling mechanism must be given the location of the sensing point SP, the tubular geometry of each applicable conductor 31 and the material resistance of each applicable conductor 31 to thereby estimate the location of contact point(s) CP as known in the art. This may be further improved by performing calibrations to refine the indicated locations for the given resistance. This location estimation of the contact point(s) CP facilitates a more precise execution of the responsive action(s) of stage S74, such as, for example, a more precise visualization of the location of the physical contact between contact detection tube 20′ and object 80 as shown in FIG. 7 and a more precise retraction, advancement and/or rotation of contact detection tube 20′ away from object 80.

For either mode as related to stage S72, two or more electrodes 30 may be integrated in contact detection tube 20′ as previously stated herein. In such cases, the general measuring mode will be able to provide an improved approximation of the location of the contact point CP, compared to the single electrode embodiment, since the overall path is first broken down into the exposed segments per tube, narrowing the choices for location.

Similarly, the specific measuring mode can calculate the distance between the stored location of the sensing point SP for each electrode 30 and the contact point CP based on the resistance measured for the length of tube, using the geometry of the tubular wall 21. Nonetheless, in practice, the detection sensitivity of contact detection tube 20′ of the present invention will be dependent upon the intended application of contact detection tube 20.

For the specific measuring mode as related to stage S73, the impedance values can be measured experimentally prior to an application of tube 20′ and used to narrow the estimated location of any physical contact between 20′ and an electrically conductive object.

Also in practice, the present invention does not impose any limitations or restrictions to the structural configuration of a contact detection tube in terms of the cross-sectional shape and dimensions of the contact detection tube. Significant features of the contact detection tube of the present invention are a portion or an entirety of the exterior surface of the tubular wall being electrically conductive and the working channel defined by the interior surface of tubular wall being electrically insulated from the exterior surface of the tubular wall. Nonetheless, to further facilitate an understanding of the present invention, FIGS. 8-15 illustrate four (4) exemplary embodiments of a contact detection tube of the present invention.

Referring to FIGS. 8A-8D, the illustrated contact detection tube has a tubular electric conductor 131 (e.g., Nitinol or copper) having an interior surface coated by an electric insulator 132 (e.g., Teflon, polymers such as polycarbonate) defining a working channel 124, wherein the interior surface and distal end of the electric conductor 131 are covered with the electric insulator 132. As shown in FIG. 9A, a contact sensing device 61 is electrically connected to a proximal end of conductor 131 whereby the impedance of conductor 131 relative to the proximal end ranges from a minimal impedance Ωₘᵢₙ to a maximum impedance Ωₘₐₓ at the distal end of conductor 131. This facilitates an impedance estimation Ωₑₛₜ by device 61 upon an electrically conductive object 81 (e.g., biological tissue or a medical instrument/tool) physically contacting conductor 131 as exemplarily shown in FIG. 9B.

Referring to FIGS. 10A-10D, the illustrated contact detection tube has a tubular electric insulator 232 (e.g. a polymer, including polycarbonate or Teflon) having an interior surface defining a working channel 224, and an electric conductor 231 (e.g., Nitinol or copper) coated on the exterior surface of electric insulator 232. As shown in FIG. 11A, contact sensing device 62 is electrically connected to a proximal end of conductor 231 whereby the impedance of conductor 231 relative to the proximal end ranges from a minimal impedance Ωₘᵢₙ to a maximum impedance Ωₘₐₓ at the distal end of conductor 231. This facilitates an impedance estimation Ωₑₛₜ by device 62 upon an electrically conductive object 82 (e.g., biological tissue or a medical instrument/tool) physically contacting conductor 231 as exemplarily shown in FIG. 11B.

Referring to FIGS. 12A-12D, the illustrated contact detection tube has a tubular electric insulator 332 (e.g., polymer such as polycarbonate, rubber or Teflon) having an interior surface defining a working channel 324, and four (4) electric conductor wires 331a-331d (e.g., Nitinol or copper) embedded on the exterior surface of electric insulator 332. As shown in FIG. 13A, a contact sensing device 63 is electrically connected to a proximal end of each conductor 331a-d whereby the impedance of each conductor 331a-d relative to their proximal end ranges from a minimal impedance Ωₘᵢₙ to a maximum impedance Ωₘₐₓ at the distal ends of conductors 331a-d. This facilitates an impedance estimation Ωₑₛₜ by device 63 upon an electrically conductive object 83 (e.g., biological tissue or a medical instrument/tool) physically contacting one the conductors 331a-d as exemplarily shown in FIG. 13B.

Alternatively, in FIGS. 13A and 13B, it may be desirable to test whether two of the wires are in contact with an object. This may happen if the object 83 crosses conductor wires 331a to 331b, which might be situated near each other for example. In this situation, the patient is no longer the ground, but rather the current runs along conductor wire 331 a and back through conductor wire 331b to contact sending device 63, for example. The resistance will also give a better estimate of the contact point, since both conducting paths are better than the human body.

Referring to FIGS. 14A-14D, the illustrated contact detection tube has a tubular electric insulator 432 (e.g., polymer such as polycarbonate, rubber or Teflon) having an interior surface defining a working channel 424, and four (4) electric conductors 431a-431d (e.g., Nitinol or copper) patterned on the exterior surface of electric insulator 432 with associated contact leads embedded within insulator 432. As shown in FIG. 15A, a contact sensing device 64 is electrically connected to a proximal end of each conductor 431a-431d whereby the impedance of each conductor 431a-431d relative to their proximal end ranges from a minimal impedance Ωₘᵢₙ to a maximum impedance Ωₘₐₓ at the distal ends of the conductors 431a-431d. This facilitates an impedance estimation Ωₑₛₜ by contact sensing device 64 upon an electrically conductive object 84 (e.g., biological tissue or a medical instrument/tool) physically contacting one the conductors 431a-431d as exemplarily shown in FIG. 15B. Based on the selected contact, such as conductor 431b as shown, the system can compute or (in particular, in the event of an open circuit or substantial increase in impedance) report the approximate location. A patterned set of conductors 431a-431d may be provided on the contact detection tube to allow for identification of locations as the conductors 431a-431d are contacted by objects or tissue or lose contact as the contact detection tube(s) are extended.

In practice, one or more tubes 20′ are employed within a contact detection system of the present invention to enable a medical tool to reach a target location within a body while minimizing, if not eliminating, any damage to the internal tissue of the body. An exemplary cannula based contact detection system will now be described herein to facilitate an understanding of contact detection systems of the present invention. Specifically, from this description, those having ordinary skill in the arts will appreciate how to construct and use other contact detection systems based on cannula tubes, nested cannulas, active cannulas, a catheter tube, an endoscopic tube, etc.

FIG. 16 illustrates a cannula based contact detection system of the present invention employing four (4) nested tubes 20′, a driving mechanism 90, a contact sensing device 100, and a controlling mechanism 110. In one embodiment, driving mechanism 90 facilitates a manual advancement and/or rotation of tubes. In an alternative embodiment, driving mechanism 90 provides mechanical guides, such as a track for precise manual advancement. In a further embodiment, tubes 20′ may be advanced electro-mechanically under computer control of driving mechanism 90. Each conductor 31 of tube(s) 20′ is electrically connected to contact sensing device 100 via respective conducting channels 91 of driving mechanism 90.

In a manual version of the system, a user manually operates driving mechanism 90 to extend and rotate as needed each contact detection tube 20′ to reach a target location within a body. As the user is operating driving mechanism 90, contact sensing device 60 provides continuity signals CS₁-CS₄ indicative of the contact status of tubes 20′ to controlling mechanism 110. Controlling mechanism 110 may be a stand-alone device communicating with other system(s) having adaptive-planning and/or imaging capabilities or be integrated within such system(s). In either case, controlling mechanism 110 provides audible and/or visual feedback to the user whenever one or more of the continuity signals CS₁-CS₄ indicate the respective tube(s) 20′ are in a closed state representative of physical contact between the tube(s) 20′ and the internal body tissue of the body or another electrically conductive object within the body.

In the manual case, for applications where such physical contact is to be avoided, the user appropriately responds to the feedback by retracting, advancing and/or rotating the tube(s) 20′ as needed to change the contact status of the physically contacting tube(s) 20′ to a open state representative of the absence of physical contact between the tubes 20′ and the internal body tissue of the body or another electrically conductive object within the body.

In the manual case, for applications where such physical contact is essential to the application, the user appropriately responds to the feedback by retracting, advancing and/or rotating the tube(s) 20′ as needed to maintain or reestablish if necessary the contact status of the physically contacting tube(s) 20′ to the closed state representative of the physical contact between the tubes 20′ and the internal body tissue of the body or another electrically conductive object within the body.

In an automatic version of the system, controlling mechanism 110 provides driving signals DS₁-DS₄ to driving mechanism 90 to thereby extend, retract and rotate as needed each contact detection tube 20′ to reach a target location within a body. As controlling mechanism 110 is controlling driving mechanism 90, contact sensing device 100 provides continuity signals CS₁-CS₄ indicative of the contact status of tubes 20′ to controlling mechanism 110. Controlling mechanism 110 provides DS₁-DS₄ that can either be electronic signals or encoded signals, or programmatic commands. Even though in this example, there are four (4) inputs CS₁-CS₄ and four (4) output driving signals DS₁-DS₄ for controlling mechanism 110, optionally provides audible and/or visual feedback to a user of the system whenever one or more of the continuity signals CS₁-CS₄ indicate the respective tube(s) 20′ are in a closed state representative of physical contact between the tube(s) 20′ and the internal body tissue of the body or another electrically conductive object within the body.

The continuity signals CS₁-CS₄ may be used by the controlling mechanism 110 in many ways. For example, the controlling mechanism 110 may generate a reflex response much like humans, which is quick to compute and perform. An example reflex is to retract the tube corresponding to the detected continuity signal. Alternatively, positions based on the surface pattern of the related signal or the derived position from electrical properties can be used to estimate the physical contact. If converted into a particular configuration of the tube set for example, location, based on the particular signal pattern n accordance with an adaptive-planning or reflex control scheme, controlling mechanism 110 provides driving signals DS₁-DS₄ to driving mechanism 90 to retract, advance and/or rotate the tube(s) 20′. Complex decisions may be made by the adaptive planner to determine the appropriate response. In some cases, as needed to change the contact status of the physically contacting tube(s) 20′ to a open state representative of the absence of physical contact between the tubes 20′ and the internal body tissue of the body or another electrically conductive object within the body.

In practice, the structural configuration and operational specification of driving mechanism 90, contact sensing device 100 and controlling mechanism 110 is dependent upon the application of tubes 20′ as would be appreciated by those having ordinary skill in the art. Additionally, the electrically conductive object must be grounded in order for the contact status of each contact detection tube 20′ to be accurately detected by device 100.

FIG. 17 illustrates an exemplary embodiment 101 of device 100 employing four (4) current meters 102 and a voltage source 103 (e.g., order of 1-10 mV rms) in the case where the electrically conductive object 85 is independently grounded. For example, object 85 is internal body tissue of a patient, ground connection is preferably established by a skin electrode placed on the patient as close as possible to the working area of conductors 31. By further example where object 85 is a medical tool external to tubes 20′, the medical tool must be electrically conductive on its exterior and electrically connected to ground. As exemplarily shown, upon one or more of conductors 31 physically contacting object 85, a continuity current I_{C} will continually flow from voltage source 103 sequentially throw a respective current meter 102(1)-(4), a respective conductor 31 and object 85 to ground. Controlling mechanism 110 (FIG. 16) utilizes continuity current I_{C} as detection signal DS indicating a closed state of the respective tube(s) 20′ and takes steps to implement responsive action(s) in dependence of operating in the general sensing mode or the specific sensing mode.

FIG. 18 illustrates an exemplary embodiment 101 of device 100 employing three (3) current meters 102 and voltage source 103 for the case where a portion 86a of an electrically conductive object (e.g., skin adjacent an incision in a body) is permanently connected to a grounded conductor 31(1). This eliminates the need for the electrically conductive object to be independently grounded. As exemplarily shown, upon one or more of conductors 31(2)-31(4) physically contacting an internal portion 86b of electrically conductive object, continuity current I_{C} will continually flow from voltage source 103 sequentially through a respective current meter 102, a respective conductor 31, electrically conductive object 86, conductor 31(1) to ground. Again, controlling mechanism 110 (FIG. 14) utilizes continuity current I_{C} as detection signal indicating a closed state of the respective tube(s) 20′ and takes steps to implement responsive action(s) in dependence of operating in the general sensing mode or the specific sensing mode.

In practice, those having ordinary skill in the art will appreciate that, while not required, a contact detection system of the present invention should be designed in a manner that limits the flow of current through the patient's body within a safe range, such as, for example, the electrical current limit recommended by the American Heart Association. It is also desirable to limit the voltage used in a contact detection system of the present invention to the maximum amperage and expected resistance. These limitations are dependent upon the actual structural configuration of device 100, such as, for example, embodiments 101 and 102 shown in respective FIGS. 17 and 18 or a volt meter-current source embodiment as would be appreciated by those having ordinary skill in the art. The medical current limiter disclosed in U.S. Pat. No. 5,761,019 to M. W. Kroll may be used a safety basis for a contact detection system of the present invention.

Further, if the contact detection system is an automated cannula system, controlling mechanism 110 may be used as a safety by the system halting the advancement of tubes 20′ upon detection of physical contact with electrically conductive object. Concurrently or alternatively, designated tube(s) 20′ may be permitted to contact electrically conductive object 80, such as, for example, the inner-most contact detection tube 20′ shown in FIG. 16. This can be useful if the inner-most contact detection tube 20′ carries a tool for performing surgical tasks, and the other tubes 20′ are intended only for supporting the reach of the innermost contact detection tube 20′.

From FIGS. 2-18, those having ordinary skill in the art will appreciate various and numerous benefits of the present invention including, but not limited to, the use of the present invention in all applications of active or nested cannula. Specifically, the present invention was primarily conceived for minimally invasive laparoscopic surgical operations where the cannula is inserted into a large free space of a patient's body (e.g., CO₂-inflated abdomen) but can in principle be used for other cannula applications (e.g., cancerous, cardiac, vascular, gastrula, and neural applications).

While various embodiments of the present invention have been illustrated and described, it will be understood by those skilled in the art that the methods and the system as described herein are illustrative, and various changes and modifications may be made and equivalents may be substituted for elements thereof without departing from the true scope of the present invention. In addition, many modifications may be made to adapt the teachings of the present invention to entity path planning without departing from its central scope. Therefore, it is intended that the present invention not be limited to the particular embodiments disclosed as the best mode contemplated for carrying out the present invention, but that the present invention include all embodiments falling within the scope of the appended claims.

1. A contact detection method (70), comprising:
navigating a contact detection tube (20′) within an anatomical region (50) of a body, the contact detection tube (20′) including
a tubular wall (21) having an interior surface (23) defining a working channel (24), and
an electrode (30) integrated in the tubular wall (21),
wherein the electrode (30) is operable to electrically connect the contact detection tube (20′) to an electrically conductive object (41, 52) within the anatomical region (50) in physical contact with an exterior surface (22) of the tubular wall (21), and
wherein the electrode (30) is further operable to electrically isolate the working channel (24) from an electrical connection of the contact detection tube (20′) to the electrically conductive object (41, 52); and
determining an electrical contact status of the contact detection tube (20′) between an open state and a closed state,
wherein the open state is representative of a sensing of an open circuit between the contact detection tube (20′) and the electrically conductive object (41, 52); and
wherein the closed state is representative of a sensing of a closed circuit between the contact detection tube (20′) and the electrically conductive object (41, 52).

2. The contact detection method (70) of claim 1, further comprising:
identifying a location of the electrode (30) relative to the tubular wall (21).

3. The contact detection method (70) of claim 1, further comprising:
estimating a contact point between the exterior surface (22) of the tubular wall (21) and the electrically conductive object (41, 52) in dependence upon an impedance of the electrode (30).

4. The contact detection method (70) of claim 1, further comprising:
executing at least one responsive action to a closed state by electrically disconnecting the contact detection tube (20′) and the electrically conductive object (41, 52).

5. The contact detection method (70) of claim 1, further comprising:
executing at least one responsive action to a closed state by maintaining an electrically connection of the contact detection tube (20′) and the electrically conductive object (41, 52).

6. A nested cannula set, comprising:
a plurality of telescoping tubes configured and dimensioned to reach a target location relative to an anatomical region (50) of a body, wherein a contact detection tube (20′) of the plurality of tube includes
a tubular wall (21) having an interior surface (23) defining a working channel (24), and
an electrode (30) integrated in the tubular wall (21),
wherein the electrode (30) is operable to electrically connect the contact detection tube (20′) to an electrically conductive object (41, 52) within the anatomical region (50) in physical contact with an exterior surface (22) of the tubular wall (21), and
wherein the electrode (30) is further operable to electrically isolate the working channel (24) from an electrical connection of the contact detection tube (20′) to the electrically conductive object (41, 52).

7. The nested cannula set of claim 6,
wherein the contact detection tube (20′) has an electric contact status between a open state and a closed state;
wherein the open state is representative of a sensing of an open circuit between the contact detection tube (20′) and the electrically conductive object (41, 52); and
wherein the closed state is representative of a sensing of a closed circuit between the contact detection tube (20′) and the electrically conductive object (41, 52).

8. The nested cannula set of claim 6, wherein the electrode (30) includes:
a tubular electric conductor (31) forming at least a portion of the working channel (24) of the contact detection tube (20′); and
an electric insulator (32) disposed on an entirety of a conductive interior surface (23) of the tubular electric conductor (31).

9. The nested cannula set of claim 6, wherein the electrode (30) includes:
a tubular electric insulator (32) forming at least a portion of the working channel (24) of the contact detection tube (20′); and
an electric conductor (31) disposed on at least a portion of an exterior surface (22) of the tubular electric insulator (32).

10. The nested cannula set of claim 6, wherein the electrode (30) provides contact location information of the electrical connection of the contact detection tube (20′) to the electrically conductive object (41, 52) as a function of at least one of a nesting order of the contact detection tube (20′), a position of the contact detection tube (20′) within the anatomical region (50) and a contact impedance of the electrode (30).

11. A contact detection system, comprising:
a contact detection tube (20′) configured and dimensioned to be navigated within an anatomical region (50) of a body, the contact detection tube (20′) including
a tubular wall (21) having an interior surface (23) defining a working channel (24), and
an electrode (30) integrated in the tubular wall (21),
wherein the electrode (30) is operable to electrically connect the contact detection tube (20′) to an electrically conductive object (41, 52) within the anatomical region (50) in physical contact with an exterior surface (22) of the tubular wall (21), and
wherein the electrode (30) is further operable to electrically isolate the working channel (24) from an electrical connection of the contact detection tube (20′) to the electrically conductive object (41, 52); and
a contact sensing device (100) in electrical communication with to electrode (30) to sense an electric contact status of the contact detection tube (20′) between a open state and a closed state,
wherein the open state is representative of a sensing of an open circuit between the contact detection tube (20′) and the electrically conductive object (41, 52); and
wherein the closed state is representative of a sensing of a closed circuit between the contact detection tube (20′) and the electrically conductive object (41, 52).

12. The contact detection system of claim 1, further comprising:
a controlling mechanism (101) in electrical communication with the contact sensing device (100) to identify a location of the electrode (30) relative to the tubular wall (21) in response to the contact sensing device (100) sensing a closed state of the contact detection tube (20′).

13. The contact detection system of claim 11, further comprising:
a controlling mechanism (101) in electrical communication with the contact sensing device (100) to estimate a contact point of the electrical connection of the contact detection tube (20′) to the electrically conductive object (41, 52) as a function of at least one of a nesting order of the contact detection tube (20′), a position of the contact detection tube (20′) within the anatomical region (50) and a contact impedance of the electrode (30).

14. The contact detection system of claim 11, further comprising:
a controlling mechanism (101) in electrical communication with the contact sensing device (100) to determine at least one responsive action to an electrical connection of the contact detection tube (20′) from the electrically conductive body (41, 52) in response to the contact sensing device (100) sensing a closed state of the contact detection tube (20′).

15. The contact detection system of claim 14, further comprising:
a driving mechanism (90) in mechanical communication with the contact detection tube (20′) and in electrical communication with the controlling mechanism (101) to execute the at least one responsive action as directed by the controlling mechanism (101).

16. A nested cannula set, comprising:
a contact detection tube (20′) including
a tubular wall (21) having an interior surface (23) defining a working channel (424), and
at least two or more conductors (431a-431d) patterned on the surface of an insulator (432) integrated in the tubular wall (21),
wherein the at least two or more conductors (431a-431d) are operable to electrically connect or disconnect the contact detection tube (20′) to or from an electrically conductive object within an anatomical region (50) in physical contact with an exterior surface of the tubular wall (21), and
wherein the at least two or more conductors (431a-431d) are operable to provide input to a contact sensing device (64) whereby a location of the contact detection tube (20′) is computed or reported.

17. The nested cannula set of claim 16, wherein the at least two or more conductors (431a-431d) form a pattern on the surface of the insulator (432).
